(19) United States
(12) Patent Application Publication
(10) Pub. No.: US 2006/0209257 A1
(43) Pub. Date: Sep. 21, 2006

(54) Integral viewing and eye imaging system for visual projection systems

(76) Inventor: Paul Bullwinkel, Stuart, FL (US)

Correspondence Address: MCHALE & SLAVIN, P.A., 2855 PGA BLVD, PALM BEACH GARDENS, FL, 33410, US

(21) Appl. No.: 11/084,596

(22) Filed: Mar. 17, 2005

(51) Int. Cl. A61B 13/00; A61B 3/14

(52) U.S. Cl. 351/210; 600/558

(57) ABSTRACT

An eye imaging system having an image viewing subsystem for interfacing with an MRI patient. The imagine viewing subsystem incorporates fiber-optic illumination and imaging subsystems that enable automatic eye imaging for eye tracking. The eye imaging system analyzes motion of an patient's eye in response to visual stimuli and includes an image conveyor subsystem, an image receiving subsystem, and an image processing subsystem. The light source utilized to form the reflected images is an independent source of illumination.

### RELATED APPLICATIONS

This application is related to U.S. Pat. No. 5,892,566 and U.S. Pat. No. 6,079,829 by the instant inventor. The disclosures of these patents are incorporated by reference.

### FIELD OF THE INVENTION

This invention is directed to eye tracking devices, particularly, to an eye tracking device suited for analyzing eye-movement of a patient undergoing diagnostic treatment within a magnetic resonance imaging (MRI) apparatus wherein such eye tracking device utilizes an out-of-band light source, and most particularly, wherein such diagnostic treatment may involve visual stimulation.

### BACKGROUND OF THE INVENTION

Monitoring of eye motion can provide a variety of information. Sleep researchers, for example, use eye motion as an indicator of various sleep stages. Also, persons with limited muscle control can use eye motion to interact with others or to control specialized equipment. Military applications that follow eye motion for targeting purposes or vehicle control have also been developed. Eye tracking devices are even used in the video game entertainment industry, where interactive environments adjust to follow the motion of a player's eye.

Another important use of eye tracking is for a patient undergoing diagnostic treatment within a MRI apparatus, where it may be necessary to know the behavior of the patient's eyes during the diagnostic procedure, particularly if the patient is viewing visual stimulii.

Many eye tracking devices monitor muscle activity to assess eye motion. For example, U.S. Pat. No. 5,517,021 discloses an eye tracking apparatus that detects bio-electromagnetic signals generated by the muscles that move an individual's eye. The signals are analyzed and corresponding control signals are produced as output. U.S. Pat. No. 5,422,689 discloses an eye tracking device that uses sensors to monitor electro-oculogram signals produced by eye motion. The sensors are coupled with a microprocessor that analyzes the signals to determine an operator's horizontal or vertical eye movement.

Other eye tracking devices rely on changes in light patterns to track eye motion. For example, U.S. Pat. No. 5,270,748 discloses an eye tracker that uses detection devices for determining the point of regard of an operator. Included conversion circuitry determines the position of fovea-reflected light, allowing computation of an individual's visual axis and the associated point of regard. U.S. Pat. No. 5,345,281 discloses a system that uses reflected infrared light to track the gaze of an operator's eye. The U.S. Pat. No. 5,345,281 system directs infrared light towards the eye and considers differences in infrared reflectivities between the pupil, iris, and sclera to compute eye position. U.S. Pat. No. 5,583,335 discloses an eye tracking system that includes an active matrix display. Pixels in the display are aligned with corresponding photodetectors. Axial light rays from the display pixels are reflected by the eye and detected by respective photodetectors. In turn, the array of photodetectors generates an eye-position-indicating electrical signal.

Although known detectors provide certain information about eye motion, they have limitations. In many cases, simple eye motion monitoring does not provide a complete picture. For example, eye tracking devices that monitor eye-moving muscles typically do not sense pupil action. Feedback regarding pupil contraction and dilation provides important cues during diagnostic medical procedures. Devices that do not track this pupil activity do not provide enough information for many types of medical tests. Other trackers, such as those that monitor reflected light, may provide some information about pupil action, but do not provide real-time visual images of the eye, itself. Without this visual image to provide context, electrical eye-position information may be hard to interpret and almost impossible to cross reference.

U.S. Pat. No. 5,892,566 teaches video tracking of the eye as embedded in a visual presentation system which relies on either ambient light or light from the visual system to illuminate the eye. The problem with this system is that signals from the visual presentation system may be temporarily intermittent or be of such a limited bandwidth as to make illumination of the eye unreliable for the purpose of forming an image of the eye or its structures. Similar problems stem from reliance on ambient light for illumination. While ambient light is usable for illumination, it is desirable to use a dedicated light source, either visible or non-visible (NV), to illuminate the eye.

As taught in U.S. Pat. No. 6,079,829, better results are obtainable when the source of illumination has a wavelength that is different than that used for the visual presentation/stimulus, thereby rendering the illumination independent of the visual signal. Infra-red, ultraviolet, or an equivalent NV portion of the light spectrum has been found to be a preferred source of dedicated illumination.

The physical and operational nature of known eye-tracking devices makes them unsuitable for use in many testing environments. For example, MRI diagnosis equipment creates an environment which makes it impossible to use known eye-tracking devices therein.

In operation, a typical MRI apparatus relies upon hydrogen protons which have a dipole movement and therefore behave as would a magnetic compass. In MRI scanning, the MRI apparatus operates as a large magnet wherein the protons align with the strong magnetic field but are easily disturbed by a brief radio frequency (RF) pulse of very low energy so as to alter their alignment. As the protons return to their orientation with the magnetic field, they release energy of a radio frequency that is strongly influenced by the biochemical environment. The released energy as detected and mathematically analyzed for display as a two dimensional proton density image according to the signal intensity of each issue.

The magnetic coils of the MRI apparatus are permanently fixed within a large structure so as to form a large magnet with a very confining entrance known as the bore. A patient is placed upon a scanner table that is integrated with the MRI apparatus and slid into the middle of the bore.

Eye tracking devices used during MRI scanning must transmit signals in a format that is not affected by the MRI apparatus. The magnetic and RF used by the MRI apparatus typically disrupt signals. Also, eye tracking devices used during MRI must not interfere with the MR imaging process, whether due to material construction or method of signal transmission. For these reasons, most conventional eye tracking devices are not suited for use in this environment.

Eye tracking devices used during MRI scanning must not interfere with the motion of an individual within the bore. Since the bore is a low-clearance area, eye tracking equipment used therein must be streamlined: bulky items simply will not fit.

Additionally, the eye tracking equipment used in MRI must not interfere with the operation of visual stimulation or patient comfort systems used as part of the diagnostic procedure.

U.S. Pat. No. 5,414,459 entitled Fiber Optic Video Glasses and Projection System addressed the need for eye stimulation within an MRI apparatus. The '459 device is formed from a shape and material of construction that are suitable for use within an MRI environment without the need for additional shielding. U.S. Pat. No. 5,892,566 teaches the integration of an eye imaging system into the system disclosed in U.S. Pat. No. 5,414,459, while U.S. Pat. No. 6,079,829 teaches the use of out-of-band illumination in the '566 device.

A limitation of the systems disclosed in these patents is that the teachings provide solutions for MR inert visual stimulation and eye tracking only for fiber optic glasses based visual presentation systems.

There is another class of visual presentation devices used for MRI that is based on the use of a projector and screen, such as taught in U.S. Pat. No. 6,774,929. These types of devices differ fundamentally from the fiber optic glasses in that the image viewed by the patient is created by direct transmission from a projector onto a screen, as opposed to the projector image being linked to an image plane by a fiber optic image guide, as in the fiber optic glasses systems. Projection based visual systems offer some distinct advantages over fiber optic glasses systems, such as ease of use and lower cost.

Eye tracking for projector based systems is also of interest for data validation and diagnostic information. Current eye tracking techniques for MR projection systems are based on the use of cameras and IR sources located outside of the magnet bore.

However, the devices of the prior art all require a very precise protocol of acquiring, aligning and focusing of the illumination beam, the camera angle and the viewable object. If any of these components is out of alignment, the tracking will not be usable. Further, because these are all separate elements any of them can be dislodged by movement of the patient or contact with the MRI.

Thus, what is needed is an eye tracking device that includes advantages of the known devices, while addressing the shortcomings they exhibit.

### SUMMARY OF THE INVENTION

Accordingly; it is an objective of this invention to provide an eye tracking device usable with projection type visual systems. The device should eliminate the alignment and focusing requirements of separate components. The eye tracking device should be impervious to magnetic environments and the output of MRI equipment. The device should not only indicate eye motion, but should also monitor pupil state. The device should be compact enough to monitor a patient located within the bore of MRI equipment and provide diagnostic feedback that allows comparison of eye movement and brain activity. Additionally, the device should be compatible with patient relaxation equipment used during an MRI session. The device should include a dedicated light source to illuminate the eye which has a wavelength that is different than that used for the visual presentation/stimulus, such as infrared illumination or the like, thereby rendering the illumination independent of the visual signal.

Another objective of the instant invention is an eye-tracking system that analyzes the motion of an individual's eye(S). As will be seen, the system is especially well-suited for analyzing the eye movement of a patient undergoing diagnostic treatment within a magnetic resonance imaging apparatus, and during which the patient is provided visual information by a video projection system.

A further objective of this invention provides a fiber optic image guide which forms an image of a patient's eye by utilizing the light delivered by a fiber optic illumination source. The illumination may utilize a wavelength which is out-of-band from that used for the visual presentation or stimulus, e.g. IR or an equivalent NV portion of the light spectrum. The fiber image guide thus conveys a real-time image of an patient's eye(s) to an included image conversion device. The conversion device, in turn, generates a electrical representation of the real-time eye image received from the fiber optic image guide. The input end of the image guide and the output end of the fiber optic illumination are integrated with the viewing device used by the patient so that when the patient adjusts the viewing device to view the projection screen, the correct image of the patient's eye(s) is automatically formed, without further adjustment of either the image guide or the illumination source.

A further objective of the invention is that because the eye imaging system moves with the patient, the patient's eye(s) can always be imaged regardless of the patient's location regardless of the patient's location within the MRI apparatus. The fiber optic nature of the image guide and the illumination system make them impervious to the magnetic and RF fields associated with the device, as well as insuring the eye tracking system will not interfere with the MRI imaging process.

The eye-tracking system of this invention locates key reference points in the digitized eye image and compares the location of those points to the position of corresponding reference points located within a control image. This comparison is made by a computer interfaced with the conversion device. The computer is directed by software that analyzes the relative positions of these reference points. Based upon the analysis, the computer software provides diagnostic feedback.

Thus, it is yet another objective of this invention to provide a viewing device used by the patient to view a projection screen, such device integrating fiber optic illumination, fiber optic imaging, and optical elements to aid in viewing the projection screen.

It is another objective of the present invention to provide an eye tracking system that is impervious to the highly magnetic and EMI-rich environment of the apparatus.

It is also an objective of the present invention to provide an eye tracking system that may be combined with diagnostic or relaxation equipment within confined environments.

It is yet a further objective of the present invention to provide an eye tracking system that selectively provides a visual image of a patient's eye for archival and/or comparison purposes.

It is also an objective of the present invention to provide an eye tracking system that allows comparison of brain activity with resultant eye motion.

It is a further objective of the present invention to provide an eye tracking system that allows diagnostic analysis of eye response to visual or other types of stimulation.

It is yet another objective of the present invention to provide an eye tracking system that provides diagnostic information related not only to eye motion, but to pupil state, as well.

It is yet an additional objective of the present invention to provide an eye tracking system that provides an improved image of the eye and its structures by including a source of illumination which is independent of the visual signal to provide reliable and repeatable illumination conditions.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWING

FIG. 1 is an exploded pictorial view of the eye tracking system of the present invention;

FIG. 2 is a pictorial representation of the viewing device used by a patient; and

FIG. 3 is an pictorial view of the device showing coaxial eye imaging and eye illumination.

### DETAILED DESCRIPTION OF THE PREFERRED EMBODIMENT

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown in the drawings and described in the specification.

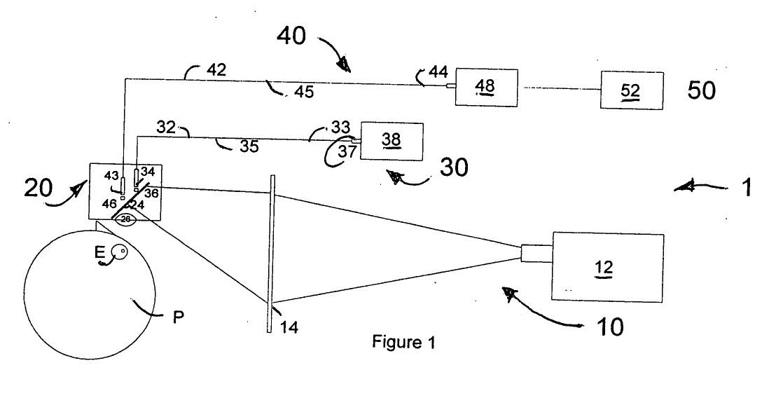

Now with respect to FIG. 1, the fiber optic eye tracking system 1 of the present invention is shown. The present eye tracking system 1 includes five cooperative subsystems: a visual stimulation subsystem 10, an illumination subsystem 30, a viewing subsystem 20, an image conveyor subsystem 40 and an image processing subsystem 50.

The visual stimulation subsystem, the illumination subsystem, the imaging receiving subsystem and the viewing subsystem are made from non-magnetic materials and are inert to the electromagnetic forces produced during MRI imaging. Additionally, the subsystems do not produce any interference with the imaging process. These subsystems may be used inside the bore of an MRI apparatus or unshielded within the MRI environment.

In this embodiment, the visual stimulation subsystem 10 includes a video output device 12 interfaced with a rear projection screen 14 and the viewing subsystem 20. This allows a visual stimulation picture to be projected on the screen 14 with the patient's view optically redirected by mirror 24 in viewing subsystem 20 to focus on the screen.

The viewing subsystem 20 integrates the mirror 24 with the output end 34 of the illumination fiber guide 32 and the input end 43 of the conveyor image guide 42. The mirror 24 is of such material or mechanical construction that allows the out-band illumination from fiber end 34 to pass through the mirror without interfering with the viewing of the visible light image, as well as allowing an uncompromised view of the patient's eye(s) by the image guide end 43. The advantage of this system is that eye imaging is independent of the patient's optically redirected view or motion of the patient's head, with the additional advantage of providing immediate eye tracking information as soon as the mirror 24 has been positioned, either by the patient or external direction, for viewing of the projection screen 14.

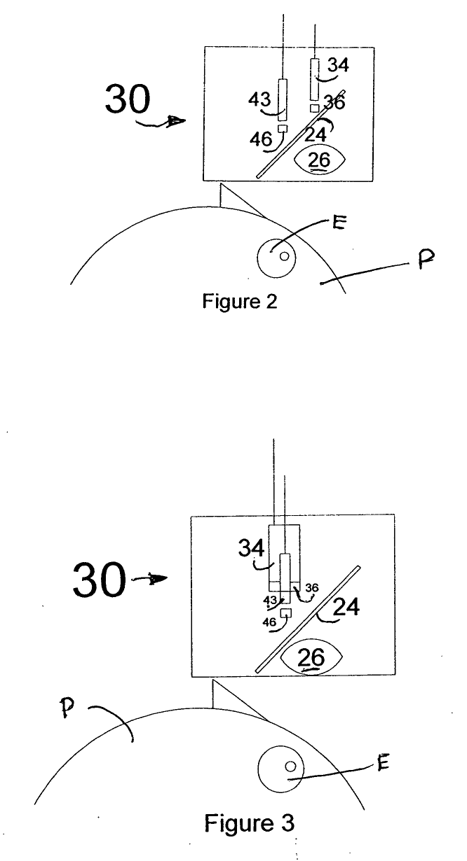

As shown in FIG. 2, the viewing subsystem 20 contains the viewing mirror 24, the end of the illumination guide 34, and the end of the image guide 43. The viewing subsystem may or may not have an optical lens for viewing the screen 14 and the viewing subsystem may or may not be supported by the patient. For example, a monocular viewing device or a binocular device could be attached directly over the patient's eye(s) by tape, headband, ear piece, nose clamp or other suppport. Alternatively, the viewing device could be moveably mounted within the MRI apparatus in close proximity to the patient's eyes.

The illumination subsystem 30 includes a flexible fiber optic guide 32 having a first end 33 in optical communication with the second end 34. The first end and the second end are spaced apart by a guide middle portion 35. The first end 33 is optically coupled at 37 to an out-of-band light source 38 which is coupled to a power source for generation of light therefrom. The second end of the guide may utilize an optical element 36 to properly distribute the illumination. The second end 34 is integrated with mirror 24 such that the NV output of the guide is directed toward a selected region of the eye, even as the viewing mirror 24 is adjusted or the patient moves his head.

Depending on the type of eye tracking used, the fiber optic illumination guide 32 or the light output from that guide may be coaxial with the image conveyor guide 42 or the image input to that guide, as shown in FIG. 3.

The image conveyor subsystem 40 delivers an electrical representation of the optically transferred real time image of the patient's eye to the image processing subsystem 50. A copy of the original and digitized images may be stored for later use as a control image. The image processing subsystem analyzes the electrical representation and generates relevant feedback.

The image conveyor subsystem 40 includes a flexible fiber optic image guide 42 having a first end 43 in optical communication with a second end 44. The first end 43 and second end 44 are spaced apart by an image guide middle portion 45. The first end 43 is directed at the patient's eye 18 during a MRI session. The first end 43 is adjustably attached to the image viewer frame and optically coupled to the patient's eye(s) such that as the mirror 24 is adjusted or if the patient moves his head, the patient's eye motion will still be tracked accurately.

The image conveyor subsystem includes a video camera 48 interfaced with the fiber optic image guide second end 44. Because the fiber optic image guide first and second ends, 43 and 44, are in optical communication, the fiber optic image guide acts as a flexible lens extension for the video camera 48. As a result, the fiber optic image guide 4 conveys a real-time eye image to the video camera 48. The video camera 48 creates an electrical representation of the transmitted real-time image and forwards the resulting electrical representation to the image processing subsystem 50.

The image processing subsystem 50 includes a computer 52 interfaced with the video camera 48. The computer 52 receives electrical output from the video camera 48 and performs operations directed by included computer hardware and software. More specifically, the video camera 48 forwards an electronic representation of the eye image to the computer 52, where the included hardware/software directs the computer to process the electronic eye image. In one embodiment, the software analyzes the digitized image of the eye and compares the location of a first reference point therein, with the location of a corresponding second reference point located in a control image. The control image may be a previously-stored image of the patient's eye E or some other suitable image. After comparing and tracking the location of corresponding reference points, the software produces diagnostic feedback. This feedback includes graphs, stimulus time/eye position charts, and a visual display of the current and/or control images of the eye E. The feedback allows a technician to make patient assessments. The feedback can also be used to control and adjust the viewing mirror 24 and optics 26.

Although the invention has been described in terms of a specific embodiment, it will be readily apparent to those skilled in this art that various modifications, rearrangements and substitutions can be made without departing from the spirit of the invention. The scope of the invention is defined by the claims appended hereto.

What is claimed is:

1. In an eye imaging system for analyzing motion of a patient's eye in response to projected visual stimulation, said system comprising an independent illumination subsystem having a flexible fiber optic guide with a first end in optical communication with an out-of-band illumination source, said first end in optical communication with a second end, said second end arranged to illuminate the eye and its structures, an image conveyor subsystem having a flexible fiber-optic image guide with a first end in optical communication with a second end, said first end positioned at a selected location with respect to the eye, said second end of said fiber-optic image guide conveying a real-time image of the eye to a camera, an image processing subsystem processing the output of said camera to provide feedback, the improvement comprising a viewing device, a means in said viewing device to optically acquire the projected stimulii, said first end of said fiber-optic image guide and said second end of said illumination fiber-optic guide movable attached and optically arranged within such viewing device whereby reliable and repeatable illumination and eye imaging conditions are automatically achieved.

2. The eye-tracking system of claim 1, including a visual stimulation subsystem including a projector and a projection screen for conveying said visual stimulii.

3. The eye-tracking system of claim 2, including a mirror mounted in said viewing device to redirect the visual stimulii from said screen to the patient's eye.

4. The eye-tracking system of claim 1 including said illumination subsystem and said image conveyor subsystem being automatically optically associated with the eye upon acquiring said stimulii.

5. The eye-tracking system of claim 4, wherein said illumination subsystem includes an independent illumination source.

6. The eye tracking system of claim 1, wherein said illumination subsystem includes an independent illumination source in the infrared wavelength range.

7. The eye tracking system of claim 1, wherein said illumination subsystem includes an independent illumination source remote from said viewing device.

8. The eye tracking system of claim 1, wherein said illumination subsystem includes an independent illumination source in said viewing device.

9. The eye tracking system of claim 1, wherein said image conveyor subsystem includes a fiber optic image guide and said illumination subsystem are coaxial.

10. The eye tracking system of claim 1, wherein said viewing device further comprises an inclined mirror, a screen is optically associated with said inclined mirror, said screen adapted for showing visual images, whereby a patient's eye tracks said visual images.

11. The eye tracking system of claim 10, wherein the improvement further comprises said screen being a video screen and said visual images are produced by a video projector.

12. The eye tracking system of claim 10, wherein said image conveyor subsystem has a fiber optic image guide and said illumination subsystem is coaxial therewith.

13. An eye tracking system for use in an RMI environment comprising, said system including
an image conveying subsystem including a flexible image guide having a first end in optical communication with a second end, said first end positioned at a selected location with respect to the eye for tracking eye movements,
said second end of said fiber-optic image guide cable for conveying a real-time image of the eye to an image processing subsystem adapted to produce and record a electronic representation of said real-time image of the eye;
an independent illumination subsystem constructed and arranged to illuminate the eye and its structures and effective to form a reflected image of the eye and its structures;
an image projection subsystem including a projection screen for conveying visual stimulii;
an image viewing subsystem having a frame, said first end of said fiber-optic image guide attached to said frame, a window in said frame conducting said light to illuminate the eye, said illumination source optically connected to said window, a mirror in said frame oriented to reflect said stimulii to the eye; and
said image conveyor subsystem, said independent illumination subsystem, said image projection subsystem and said image viewing subsystem are inert to the RMI environment;
whereby the eye movements in response to stimulii can be recorded without interference to MRI imaging.

14. An eye tracking system inert to an RMI environment of claim 13 further comprising said illumination subsystem producing out-of-band light imperceptible to the patient.

15. An eye tracking system inert to an RMI environment of claim 14 further comprising portions of said illumination subsystem being coaxial with said image guide.

16. An eye tracking system inert to an RMI environment of claim further comprising said image guide including fiber optics.

17. An eye tracking system inert to an RMI environment of claim 13 further comprising said image receiving subsystem including an archive in which the RMI imaging is correlated with said eye movements and said visual stimulii.
